(19) United States
(12) Patent Application Publication
(10) Pub. No.: US 2008/0097215 A1
(43) Pub. Date: Apr. 24, 2008

(54) Ultrasonic probe with finger shelf

(76) Inventors: Mark A. Kliewer, Madison, WI (US); Timothy Walker, Verona, WI (US); Aileen M. Ferris, Verona, WI (US)

Correspondence Address: BOYLE FREDRICKSON S.C., 840 North Plankinton Avenue, MILWAUKEE, WI, 53203, US

(21) Appl. No.: 11/523,307

(22) Filed: Sep. 19, 2006

(51) Int. Cl. A61B 8/00

(52) U.S. Cl. 600/459

(57) ABSTRACT

A cuff fitting around an ultrasonic probe provides a shelf that may receive forces from the sonographer's fingers without requiring increased gripping by the sonographer.

### CROSS-REFERENCE TO RELATED APPLICATIONS

--

### STATEMENT REGARDING FEDERALLY SPONSORED RESEARCH OR DEVELOPMENT

--

### BACKGROUND OF THE INVENTION

The present invention relates to hand-held ultrasonic probes for medical imaging and, in particular, to an improved design for the outer gripping surface of such a probe.

Ultrasound medical imaging uses ultrasound sound waves to construct an image of internal body structures. During an ultrasound imaging procedure, an ultrasonic transducer (probe) is pressed against a patient to transmits ultrasound into the patient and to receive echoes that may be analyzed to produce the image. The probe is typically hand held by a sonographer to be easily manipulated to direct the ultrasound to a desired region of interest and/or to sweep the direction of the ultrasound to create a dynamic image of internal body structure.

In some obese patients, forces of up to 40 pounds of pressure are needed between the probe and the patient. Often, and especially with portable ultrasound units which are the only imaging devices available in an intensive care unit, the sonographer must assume awkward postures to reach around large or incapacitated patients. Ultrasound technologists may be required to hold and manipulate a probe against the body of a patient for long periods of time, particularly for biopsy or ablation procedures.

The probe body is normally constructed of a hard, seamless polymer shell protecting the probe electronics and allowing for easy cleaning of the probe, but making the probe difficult to grip. This problem can be exacerbated by the acoustic coupling gel normally used to increase acoustic coupling between the probe and the patient which can contaminate the surface of the probe, making it slippery. For this reason, it is known in the art to reduce the fatigue of the sonographer by placing ribs or cushioning material on the surface of the ultrasound at the probe.

In the approximately thirty years that real time ultrasonic scanning has come into prominence as a diagnostic tool, work-related injuries have become epidemic among medical sonographers. Approximately 80% of sonographers report that they have some sort of musculoskeletal complaint of the hand and wrist. Career-ending injuries due to daily stresses of sonography affect approximately 20% of the work force. The most frequent injuries are tendonitis, tenosynovitis, thoracic outlet syndrome and carpal tunnel syndrome.

### SUMMARY OF THE INVENTION

The present invention provides an improved gripping surface for a hand-held ultrasonic probe that includes a finger “shelf” that permits an axial force to be applied to the probe without a concomitant need to increase gripping force to prevent the probe from shifting in the sonographer's hand. By limiting “pinch and push” by the sonographer, it is believed that the risk of wrist and hand injuries is significantly reduced.

Specifically then, the present invention provides an ultrasound probe having an axially extending body terminating at a probe face emitting and receiving ultrasound along an axial ultrasound path. The body includes a shelf extending radially from the body by substantially no less than the width of an adult finger, providing a face abutting a forward surface of opposing fingers of a sonographer who is grasping the body, so that an axial force may be exerted on the probe by the sonographer without this force causing substantial radial outward force on the opposing fingers.

Thus, it is an object of at least one embodiment of the invention to reduce the grip force necessary to hold an ultrasonic probe in position.

The face may extend at an angle no less than 90 degrees from an axis of the probe measured between the surface and a center of the ultrasound path.

Thus, it is an object of at least one embodiment of the invention to prevent axial force from requiring an increased gripping of the probe.

The shelf may extend more than 270 degrees about the axis of the probe.

Thus, it is an object of at least one embodiment of the invention to provide a probe that may be used at a variety of different axial angles and still provide the desired support faces.

The shelf may include an outer cushion at the face.

Thus, it is an object of at least one embodiment of the invention to compensate for the increased pressure between the shelf and the sides or top of the fingers as decreased gripping force reduces force on the pads of the fingers.

The probe may include a second shelf axially displaced from the first shelf, the second shelf extending radially from the body by substantially the width of an adult finger, providing a second face abutting a front surface of at least one finger of a sonographer.

Thus, it is an object of at least one embodiment of the invention to distribute the forces among multiple shelves and fingers decreasing the pressure on individual fingers.

The shelf may include a first portion having an inner surface conforming to an outer shell of the probe and removable therefrom, and a second cushion portion attached to an outer surface of the first portion, having a greater plasticity than the first portion.

Thus, it is an object of at least one embodiment of the invention to provide a means for removably installing the desired gripping surface onto a standard hard body probe.

These particular objects and advantages may apply to only some embodiments falling within the claims and thus do not define the scope of the invention.

### BRIEF DESCRIPTION OF THE DRAWINGS

FIG. 1 is a side elevational view of a prior art ultrasound probe showing an outward splaying force on the sonographer's fingers caused by axial force applied to the probe, such splaying force requiring increased gripping force to prevent the sonographer's fingers from slipping along the probe;

FIG. 2 is a figure similar to FIG. 1, but of the present invention, showing a cross-section of a detachable cuff that adds finger shelves to the probe eliminating the splaying force;

FIG. 3 is a top plan view of the cuff of FIG. 2, before installation on a probe, showing a split form that may be clamped together about the probe and retained by a strap;

FIG. 4 is a fragmentary, detailed view of FIG. 3, also showing the probe in partial cross-section, and illustrating an inner surface of the cuff that grips the probe and an outer cushion surface contacting the fingers; and

FIG. 5 is a side elevational view of an alternative embodiment of the cuff of FIG. 2 having second shelf for providing additional points of contact between shelves and fingers.

### DETAILED DESCRIPTION OF THE PREFERRED EMBODIMENT

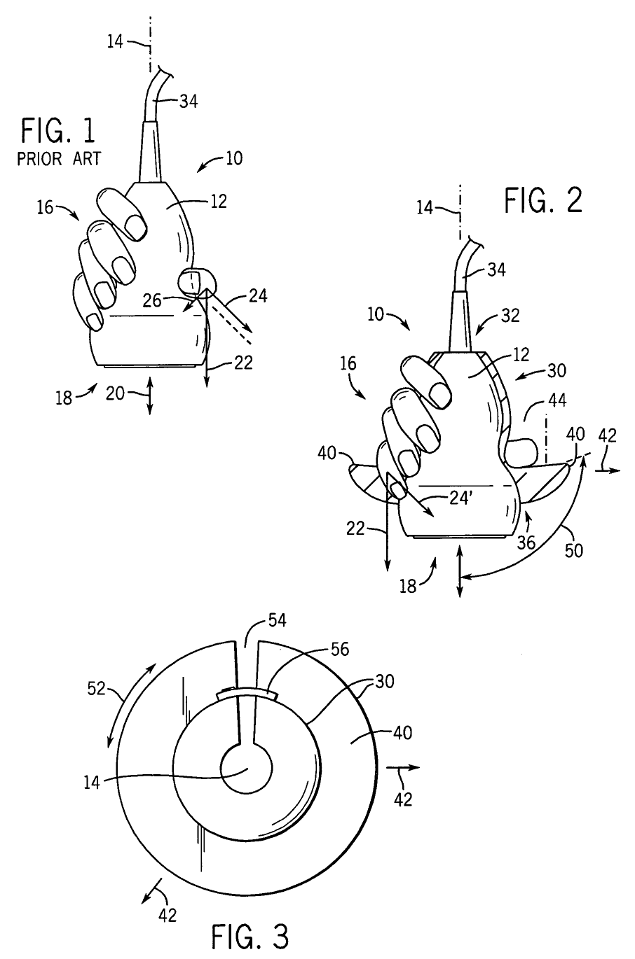

Referring now to FIG. 1, a conventional ultrasonic probe 10 may include a hard polymer body 12 extending along an axis 14 and grippable by the fingers 16 of the sonographer.

A front face 18 of the ultrasonic probe 10 emits and receives ultrasonic energy along ultrasound path 20 and is pressed against the surface of a patient with an axial force 22. Because the outer surface of the body 12 is generally parallel to or slopes along the axis 14, the axial force 22 causes an outward splaying 24 of the sonographer's fingers 16 requiring an countervailing inward gripping pressure 26 to prevent loss or slippage of the probe 10.

Referring now to FIG. 2, a first embodiment of the present invention provides a cuff 30 that may surround the body 12 of the probe 10. The cuff 30 provides a rearward opening 32 through which a cable 34 connecting the probe 10 to an ultrasound machine (not shown) may pass, and a forward opening 36 through which the front face 18 of the probe may extend.

Importantly, the cuff 30 provides for a shelf 40 extending along a radial direction 42 generally perpendicular to the axis 14. The shelf 40 extends from the adjacent outer surfaces of the cuff 30 by an amount no less than the width 44 of an average adult finger and allows force to be exerted by the sonographer through the sides or tops of the sonographer's fingers 16 as well as through the inner opposed pads of the fingers 16. The term fingers, as used herein, also includes thumbs.

Ideally, an angle 50 between the rear surface of the shelf 40 contacting the front surface of the sonographer's fingers 16 and the ultrasound path 20 will be greater than 90 degrees. In this case, the force 22 applied to axially press the probe 10 against the patient will create no net outward splaying force 24, and may even produce an inward clasping force 24′, such as does not require increased gripping by the sonographer. This reduced need for gripping force while pressing on the probe may reduce occupational injuries from gripping, pinching, or pulling-type motions.

Referring now to FIG. 3, the shelf 40 may extend radially outward around the axis 14 of the probe 10 (not shown in FIG. 3) to subtend over 270 degrees in a circumferential direction 52 about the cuff 30 to provide for support at a variety of different rotational angles of the ultrasonic probe 10 about its axis. An axially extending slot 54 may be cut through the wall of the cuff 30 from the opening 32 to the opening 36 allowing the cuff to be split and the ultrasonic probe 10 to be inserted therein. A gripping tab 56, for example, a strap with hook and loop fasteners positioned on both the outer surface of the cuff 30 and inner surface of the gripping tab 56, may be used to cinch the cuff 30 around the probe 10 during use.

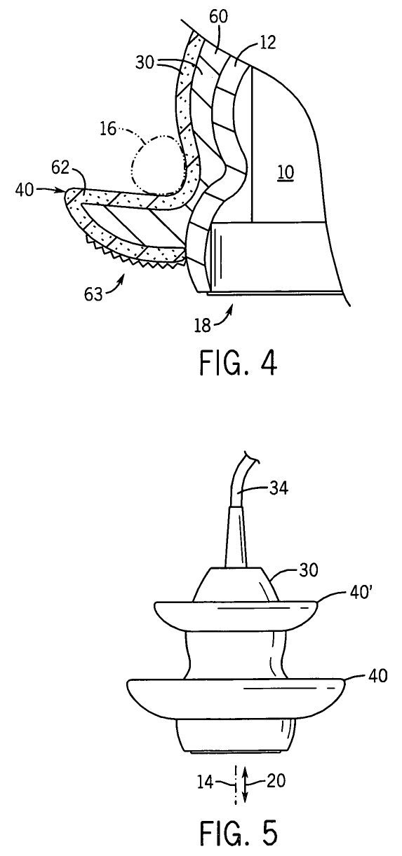

Referring now to FIG. 4, the cuff 30 may have an inner layer 60 of a hard polymer material conforming to the outer surface of the body 12 of the ultrasonic probe so as to resist axial slippage between the inner layer 60 and the body 12 when inner layer 60 is cinched to body 12. Inner layer 60 may in turn be coated with a cushioning material, for example, a closed cell foam, to create an outer cushion layer 62 that is more deformable, hence more plastic, than the inner layer 60, the latter which while somewhat elastic, tends to retain its shape. The closed cell foam may allow easy cleaning of the cuff 30, while removal of the cuff 30 allows easy cleaning of the probe 10.

The plasticity of the outer cushion layer 62 reduces the pressure on the sonographer's fingers to prevent possible discomfort as may be caused by a pressing on the forward faces of the fingers 16 rather than on their inner opposed surfaces which lightly grip the ultrasonic probe 10. The forward surface of the fingers 16 will typically be the sides of the fingers 16 as face the front face 18 surface of the probe 10, but may in fact include the tops or knuckles of the fingers 16 depending on how the cuff 30 is held.

The lower surface 63 may be displaced back from the front face 18 of the probe so as to prevent its contact directly with skin of the patient, such as may increase the forces necessary to press the ultrasonic probe 10 in against the patient. Further, the lower surface 63 may be provided with an absorbing or barrier pattern to reduce the migration of gel up into the area of the fingers 16.

Referring now to FIG. 5, it will be understood that additional axially displaced shelves 40′ may be positioned on the cuff 30 that may allow for positioning of different fingers 16 against the shelves 40 and 40′ further dividing the axial force 22 among the fingers 16. The shelves 40′ need not extend continuously around the cuff, but may be placed at locations where they may best fit between the fingers 16 of the sonographer.

While the cuff is shown as a replacement item, it will be recognized that a similar design can be incorporated directly into the housing of the ultrasonic probe by the manufacturer.

It is specifically intended that the present invention not be limited to the embodiments and illustrations contained herein, but include modified forms of those embodiments including portions of the embodiments and combinations of elements of different embodiments as come within the scope of the following claims.

We claim:

1. An ultrasound probe comprising an axially extending body terminating at a probe face emitting and receiving ultrasound along an axial ultrasound path, the body including a shelf extending radially from the body by substantially no less than a width of an adult finger to provide a face abutting a forward surface of opposing fingers of a sonographer who is grasping the body, so that axial force may be exerted on the probe by the sonographer without this force causing substantial radially outward force on the opposing fingers.

2. The ultrasound probe of claim 1 wherein the face of the shelf extends at an angle no less than 90 from an axis of the probe measured between the face and a center of the ultrasound path.

3. The ultrasound probe of claim 1 wherein the shelf extends more than 270 degrees about the axis of the probe.

4. The ultrasound probe of claim 1 wherein the shelf includes a cushion at the face.

5. The ultrasound probe of claim 1 further including a second shelf axially displaced from the shelf, the second shelf extending radially from the body by substantially the width of an adult finger providing a second face abutting a front surface of at least one finger of the sonographer.

6. The ultrasound probe of claim 1 wherein the shelf includes a first portion having an inner surface conforming to an outer shell of the probe and removable therefrom.

7. The ultrasound probe of claim 6 further including a cushion attached to an outer surface of the first portion and having a greater plasticity than the first portion.

8. The ultrasound probe of claim 6 wherein the first portion provides an axial seam allowing opening of the first portion to install around the outer shell of the probe and further including a fastener closing the first portion around the outer shell when the first portion is so installed.

9. A grip sleeve for a hand-held ultrasonic probe having an axially extending body terminating at a probe face emitting and receiving ultrasound, the grip sleeve comprising:
a first portion having an inner surface conforming to an outer shell of the probe to resist axial movement therebetween when closed around the outer shell and removable therefrom when opened; and
a shelf extending radially from the first portion by substantially a width of an adult finger providing a face abutting a side surface of opposing fingers of a sonographer grasping the body, so that axial force may be exerted on the probe by the sonographer without substantial radially outward force on the opposing fingers.
